(19) United States
(12) Patent Application Publication
(10) Pub. No.: US 2009/0270594 A1
(43) Pub. Date: Oct. 29, 2009

(54) USE OF THYMOSIN ALPHA 1 FOR THE PREPARATION OF A MEDICAMENT FOR THE PREVENTION AND TREATMENT OF ALLERGIES

(75) Inventors: Luigina Romani, Perugia (IT); Francesco Bistoni, Perugia (IT); Enrico Garaci, Roma (IT); Guido Rasi, Roma (IT); Paola Sinibaldi Vallebona, Roma (IT)

Correspondence Address: ARENT FOX LLP, 1050 CONNECTICUT AVENUE, N.W., SUITE 400, WASHINGTON, DC, 20036, US

(73) Assignee: SIGMA-TAU INDUSTRIE FARMACEUTICHE, Pomezia (IT)

(21) Appl. No.: 12/447,156

(22) PCT Filed: Sep. 27, 2007
(86) PCT No.: PCT/IT07/00676

§ 371 Date: Jul. 6, 2009

(30) Foreign Application Priority Data
Oct. 27, 2006 (IT) RM2006A000583

(51) Int. Cl. C07K 14/47

(52) U.S. Cl. 530/350

(57) ABSTRACT

The invention concerns the use of thymosin alpha 1 for the preparation of a medicament for the prevention and treatment of allergies

The present invention concerns the use of thymosin alpha 1 for the preparation of a medicament for the prevention and treatment of allergies.

Allergies are an excessive reaction to substances that are not generally dangerous to man, given that they constitute a paroxitic immunitary response compared to a normal one. The body's immune system normally protects us from pathogens such as bacteria, viruses or toxic substances. An allergy is instead the reaction of a hypersensitive immune system towards non-pathogenic organisms. It is the first exposure to an allergen which causes the allergic reaction in the individual and makes him recognise the allergen every time he comes into contact with it later. The symptoms arise in the second and all subsequent exposures and strictly depend not only on the allergen concerned, but also on the part of the body affected and on the intensity of the immunitary reaction. When the allergen comes into contact with an individual's immune system, it stimulates the production of antibodies which bind to cells containing histamine. It is the production of this substance that causes the typical allergic symptoms in the patient: itching, swelling of the affected tissues, hypersecretion of mucous, and muscle spasms. The severity and variety of these symptoms is strongly subjective because it depends on the individual.

The most common allergens are food, drugs, some substances contained in cosmetics, certain metals used in jewelry or trinkets, insect bites, dust mites, pollen, moulds and pets.

Breast-fed children have a lower probability of contracting allergies, including exthma, if the mothers have not had certain foods during the breast-feeding period, such as cow milk, eggs and nuts. If an allergy arises, then only a suitable therapy and the possibility of avoiding contact with the relative allergens can reduce to a minimum a possible recurrence of the allergy crisis in future.

The symptoms may vary in intensity and typology depending on the reaction, the body area affected and on the sensibility of the patient's immune system. There are, however, some common symptoms: rhinitis, coughing, respiratory problems, increased lacrimation, itching in the contact area (eyes, nose, throat and skin in general), skin rashes, vomiting, diarrhea, headaches.

Allergic illnesses are caused by the induction of T helper cells (Th2) and IgE-specific responses for the common environmental antigens (allergens) in susceptible individuals. There is increased interest in the role of natural or induced T regulatory (Treg) cell populations in the prevention of these inappropriate immune reactions underlying the sensibilisation to allergens. Current evidence suggests that Treg can actively prevent Th2 responses to allergens present in non-atopical individuals and that their function can be weakened in allergic patients. There is evidence that the treatments can act by modulating the Treg function. Current studies aim to understand the mechanisms involved in the generation and function of allergen-specific Treg. A primary aim is to promote the development of treatment plans geared to the induction of lasting allergen-specific inhibitory mechanisms but which are, at the same time, localised and not generalised.

To date, allergies are treated with anti-histamine and anti-inflammatory drugs such as those containing cortisone or sometimes by means of homeopathic therapies. However, the treatments with some drugs can cause considerable—and sometimes serious—side effects such as immunosuppression or various metabolic disorders.

In view of the above, there is thus the evident need for new drugs for the prevention and treatment of allergies that do not envisage the disadvantages of known therapies.

The authors of the present invention have now found that thymosin alpha 1 can effectively prevent and treat allergies without causing any toxic effects for the body.

Thymosin alpha 1 (Tα1), a thymic peptide found in nature, is well known for the treatment of some viral infections both as a monotherapy and in association with IFN-α, and as an immunitary adjuvant (Goldstein A. et al., 2004 Expert Opin. Biol. Ther. 4:559-573).

Other therapeutic indications are also known about thymosin alpha 1 such as in the treatment for immunodeficiency, tumours and AIDS. Thymosin alpha 1 is also known as a modulator of the biological response in the treatment of certain viral infections in association with INF-α, and as an immune adjuvant (Goldstein A. et al., 2004 Expert Opin. Biol. Ther. 4:559-573). Recent studies have brought to light a new and unexpected role for this molecule. It has recently been demonstrated that Tα1 modulates the functioning of dendritic cells (DC) through the toll-like receptor (TLR) 9, thus acting as an endogenous regulator of the inborn and adaptive immune systems (Romani L. et al., 2004, Blood 103:4232-4239). Thanks to this functional activity on DC, Tα1 has been found to be able to induce functionally active Treg both in vitro and in vivo (Romani L, Blood 2006). These studies provide the rational premises for the use of Tα1 as an inducer of functionally active Treg. In the face of a generalised and indiscriminate suppression, such as the one achieved by current corticosteroid therapies, this approach would offer the advantage of a regulation of highly selective and specific aberrant immune reactions.

The specific object of the present invention is thus the use of Tα1 for the preparation of a medicament for the prevention and treatment of allergies in which the allergies are, for example, those caused by allergens such as substances contained in food, like ovalbumin, drugs, substances contained in cosmetics, certain metals used in jewelry or trinkets, such as nickel, insect bites, dust mites, pollen, moulds, pets, and fungi like Aspergillus fumigatus.

The present invention will now be described for illustrative purposes, but is not limited to this, according to some preferred embodiments, with particular reference to the figures in the attached drawings, wherein:

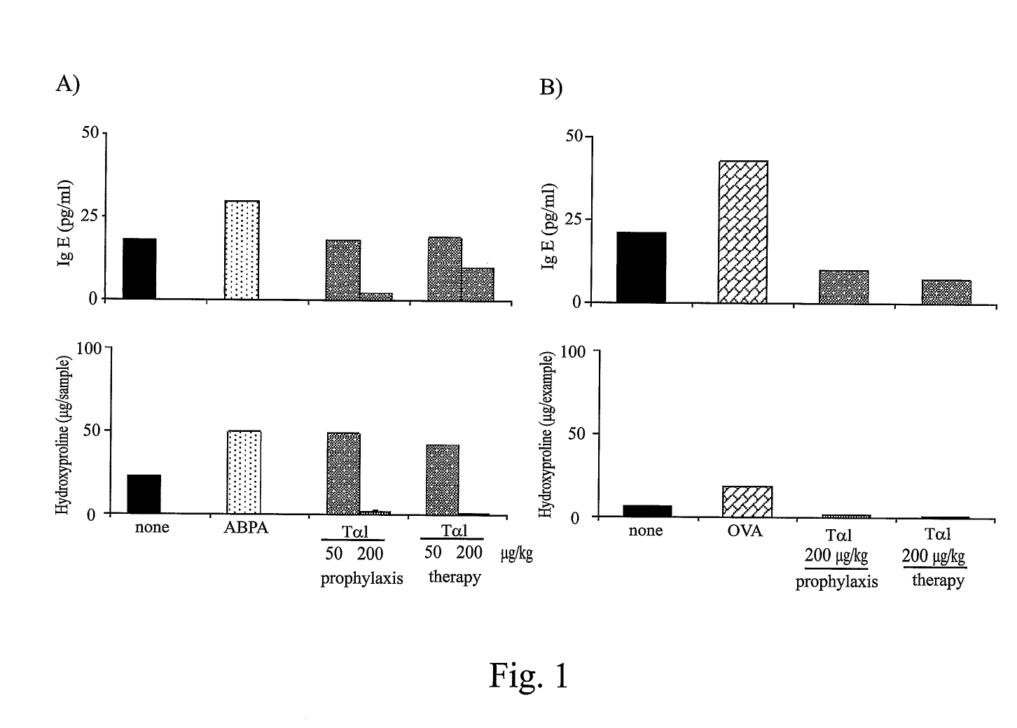

FIG. 1 shows the data on the effectiveness of Tα1 in ABPA (A) and in allergy from OVA (B). None represents the control mice.

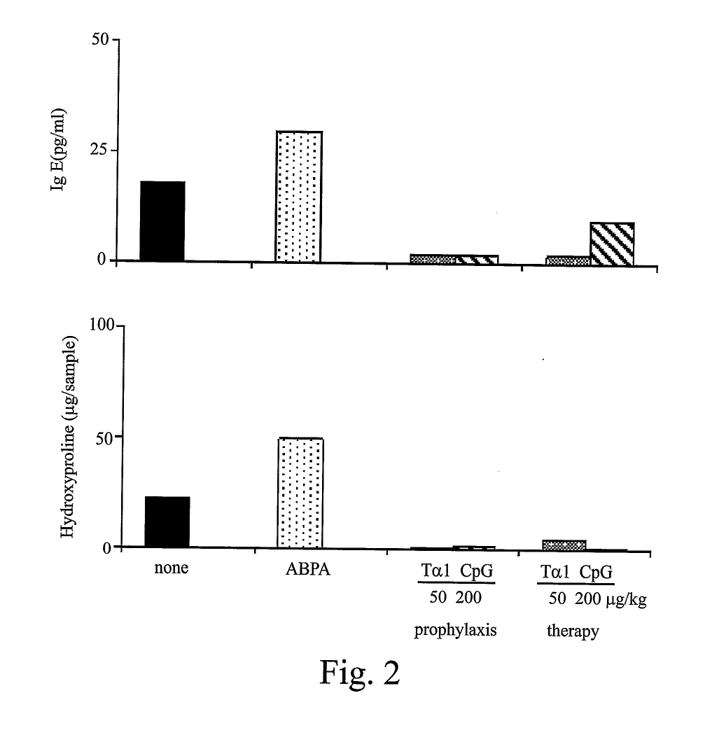

FIG. 2 shows the data on the comparison of the effectiveness of Tα1 and CpG in ABPA.

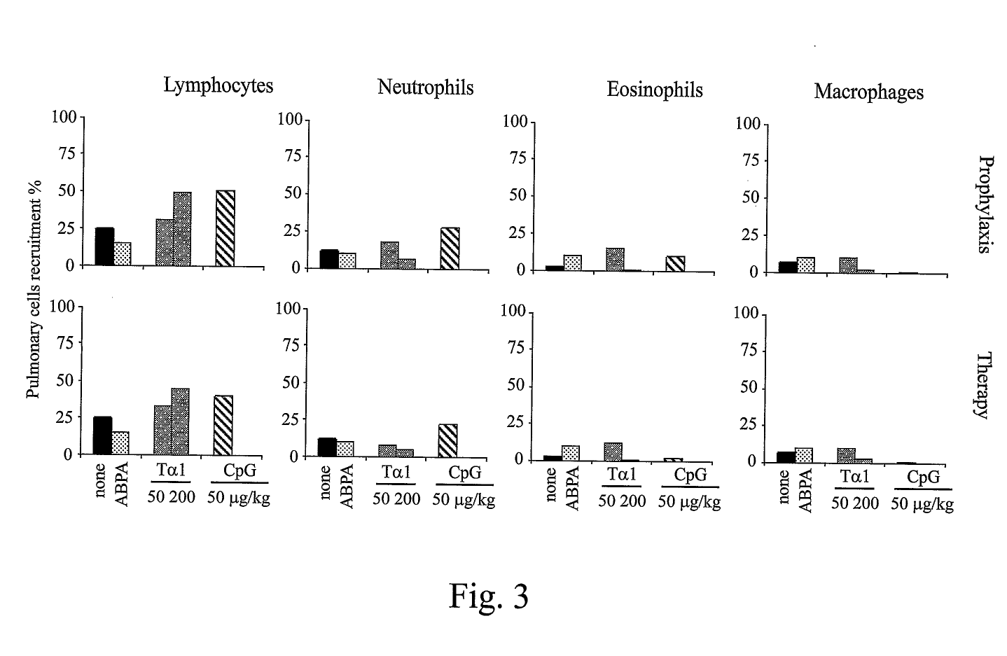

FIG. 3 shows the data on the effect of Tα1 on pulmonary lymphomonocyte recruitment in ABPA.

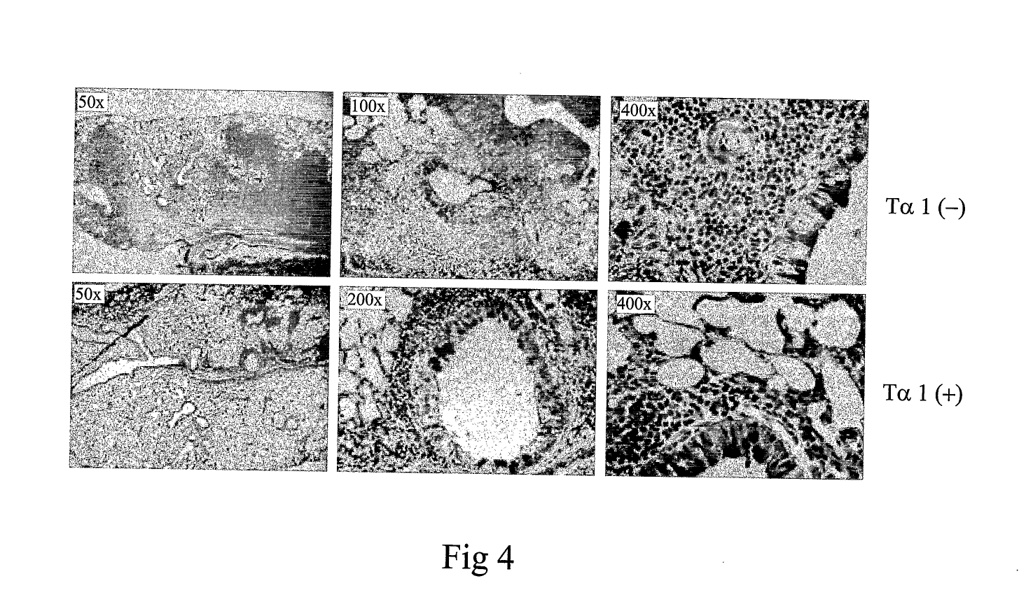

FIG. 4 shows the data on the effect of Tα1 on the local inflammatory pathology in ABPA.

### EXAMPLE 1

Evaluation of the efficacy of thymosin alpha 1 in two experimental models of allergy. A model consists of inducing an allergic state by administering chicken ovalbumin (OVA) in allergising conditions. The other consists of inducing the allergy from environmental allergens, and namely the spores of the fungus Aspergillus fumigatus.

Methodology

Allergy and Treatment

The induction of the allergic pathology due to Aspergillus (called ABPA, Allergic Bronchopulmonary Aspergillosis) and to OVA was carried out as described in table 1 (Montagnoli C, Fallarino F, Gaziano R, Bozza S, Bellocchio S, Zelante T, Kurup W P, Pitzurra L, Puccetti P, Romani L. Immunity and tolerance to Aspergillus involve functionally distinct regulatory T cells and tryptophan catabolism. J. Immunol. 2006, 176:1712-1723). The mice received an intraperitoneal (i.p.) and subcutaneous (s.c.) injection of 5 μg of A. fumigatus culture filter (CCFA) dissolved in an incomplete Freund adjuvant (Sigma) followed by two consecutive intranasal injections (one week apart) of 20 μg CCFA. One week after the last intranasal contact, the mice received 10⁷ Aspergillus conidia intratracheally (i.t.) for OVA sensibilization. The mice received 10 μg of OVA via i.p./s.c. on day 0, followed by two consecutive intranasal injections (one week apart) of 10 μg of OVA (chicken OVA grade VI; Sigma-Aldrich) together with 1 mg of Al(OH)₃ (Alum Inject; Pierce, Rockford, Ill.) as adjuvant dissolved in a sterile saline solution. The mice were analysed for inflammation and allergy parameters a week later. For the histological analysis, sections (from 3 to 4 μm) of tissues immersed in paraffin were coloured with periodical Schiff acid (PAS) to evaluate the general morphology. The lung sections coloured with PAS were examined at 50, 200 and 400 enlargements to assess the “Globet” type cells typical of the allergy.

The differential total and total lung cell counts were carried out by colouring the lung of allergic mice with May-Grünwald reagents (Giemsa Sigma) before the analysis.

Hydroxyproline Determination

The collagen levels in the lungs were determined as described (Montagnoli C, Fallarino F, Gaziano R, Bozza S, Bellocchio S, Zelante T, Kurup WP, Pitzurra L, Puccetti P, Romani L. Immunity and tolerance to Aspergillus involve functionally distinct regulatory T cells and tryptophan catabolism. J. Immunol. 2006, 176:1712-1723). The concentrations of hydroxyproline were calculated by means of a standard hydroxyproline curve (zero to 100 μg/ml).

IgE determination in the serum. Total IgE in the serum samples was determined by enzyme-linked immunosorbent assay (Montagnoli C, Fallarino F, Gaziano R, Bozza S, Bellocchio S, Zelante T, Kurup WP, Ptzurra L, Puccetti P, Romani L. Immunity and tolerance to Aspergillus involve functionally distinct regulatory T cells and tryptophan catabolism. J. Immunol. 2006, 176:1712-1723 in the serum samples was measured by enzyme-linked immunosorbent assay).

Treatment with thymosin alpha 1. Tα1 (purchased from Sigma, St. Louis, Mo., USA; product no. T3410; molecular formula C₁₂₉H₂₁₅N₃₃O₅₅) and the scrambled peptide were supplied as sterile dried powders. The powders were reconstituted in sterile water (endotoxin levels were <0.03 pg/ml, by means of standard Limulus lysate assay). Tα1 was administered at a dosage of 50 and 200 microgrammes/kg i.p. according to the design reported in tables 1 and 2, both in the ABPA model and in the allergy from OVA. Mice controls received the scrambled peptide. In selected experiments, the mice were treated for comparison with a known agent that could improve the allergic symptomatology: oligodeoxynucleotide, containing non-methylated CpG (CpG) sequences.

TABLE 1

| | Induction phase | | reaction phase | |
|---|---|---|---|---|
| Day | 0 | 7 | 14 | 21 |
| CCFA ^{a} | 5 g/i.p. + 5 g/s.c. | 20 g/i.n. | 20 g/i.n. | Aspx × 10⁷/i.t. |
| OVA | 10 g/i.p. + 10 g/s.c. | 0 g/i.n. | 10 g/i.n. | Aspx × 10⁷/i.t. |

^{a} CCFA, culture filtrate of A. fumigatus

TABLE 2

| | |
|---|---|
| Prophylaxis | Treatment |

| | | | | | |
|---|---|---|---|---|---|
| Day | 0 | 7 | 14 | 21 | 26 |

| | | | | |
|---|---|---|---|---|
| Tα1 | i.p./s.c. | i.n. | i.n. | i.p. |

FIG. 1 shows the data regarding the efficacy of Tα1 in ABPA (A) and in allergy from OVA (B). The results clearly show a drastic reduction in allergic inflammatory parameters after Tα1 treatment, and namely the local production of hydroxyproline and IgE antibodies, the mediators of allergic inflammation. The effect was dose-dependent, found at a dosage of 200 microgrammes of Tα1i and not at lower dosages, and was seen both for the prophylaxis and the actual therapy. None stands for the control mice.

FIG. 2 shows the comparative data on the effectiveness of Tα1 and CpG in ABPA. The results clearly show a comparable efficacy of Tα1 with respect to CpG in reducing the allergic inflammation parameters, both in prophylaxis and therapy. None stands for the control mice.

FIG. 3 shows the data on the effect of Tα1 on pulmonary lymphomonocyte recruitment in ABPA. The data show a drastic reduction in the recruitment of eosinophils—the cells responsible for the morbose manifestations of the allergy—by the action of Tα1, both in prophylaxis and in therapy. Here, too, the effect was comparable to that of CpG. Tα1 did not have any appreciable effects on the recruitment of other cell typologies, such as neutrophils and monocytes/macrophages.

FIG. 4 shows the data on the effect of Tα1 on local inflammatory pathology in ABPA. Different enlargements of the pulmonary sections of mice with ABPA treated with (+) or without (−)Tα1 show marked differences in terms of: i) inflammatory infiltrate, which is considerably reduced after prophylactic treatment with Tα1; ii) the presence of submucose glandular cells (called Globet cells), which are the main producers of mucous and are visible by PAS purple colouring. Treatment with Tα1 is associated with a decreased hyperplasia of these cells.

On the whole, the data suggest that Tα1 can have a marked anti-allergic effect both in the prevention and treatment of allergies.

1. Use of thymosin alpha 1 for the preparation of a medicament for the prevention and treatment of allergies.

2. Use according to claim 1, wherein the allergies are the ones caused by allergens such as substances contained in food, drugs or cosmetics, metals contained in jewelry or trinkets, insect bites, dust mites, pollen, moulds, pets and fungi.

3. Use according to claim 2, wherein the substance contained in food is ovalbumin.

4. Use according to claim 2, wherein the fungus is Aspergillus fumigatus.
